(19) United States
(12) Patent Application Publication
(10) Pub. No.: US 2010/0234714 A1
(43) Pub. Date: Sep. 16, 2010

(54) DYNAMIC BODY STATE DISPLAY DEVICE

(75) Inventors: Virginie Mercier, Eindhoven (NL); Sima Asvadi, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/377,690

(22) PCT Filed: Aug. 10, 2007
(86) PCT No.: PCT/IB07/53178

§ 371 Date: Feb. 17, 2009

(30) Foreign Application Priority Data
Aug. 17, 2006 (EP) 06119104.5

(51) Int. Cl. A61B 5/04

(52) U.S. Cl. 600/388

(57) ABSTRACT

Dynamic body state display device, which is wearable by a person on the body and which is at least partly flexible, wherein the device comprises a dynamic body state sensor for measuring the dynamic body state, a storage arrangement for storing information specifying dynamic body state ranges, a processing circuit configured to process the measured dynamic body state and determine the corresponding dynamic body state range, and an at least partly flexible structure, that can be worn on the body, wherein the at least partly flexible structure comprises a display for visually indicating said corresponding dynamic body state range.

### FIELD OF THE INVENTION

The invention relates to a dynamic body state display device.

### BACKGROUND OF THE INVENTION

Conventional dynamic body state devices, for example, heart rate devices are oftentimes provided with a relatively small screen, e.g. a LCD screen, to display information about the heart rate or other body state data. Possibly, these devices have some kind of alert function installed, for example to warn a user when his or her heart rate is above a certain limit. When such an alert occurs, a sound signal is given. These devices may be worn in noisy environments, or for example while listening to music wearing headphones. For example, exercising outdoors in urban environment provides a level of noise (bus or car traffic, shouting children, etc.) that can cover the alert sound. Also, it is common to wear headphones or listen to music through speakers, while jogging or while doing indoor fitness. For these and other reasons, the sound signals are oftentimes ignored and/or not heard.

Persons using said conventional devices while exercising are not aware of the information that is displayed, because of the small screen. Also a user may have to search the small screen or scroll through a menu to get the information that is needed. Observers of those exercising persons wearing said device cannot know what is the body state of that person as they have less probability to see the small display from far away. Said devices could for example communicate wireless with a computer such that the observer could view certain body states on a computer screen, but it still would be hard to create some kind of overview of each individual when more exercising individuals are involved. Especially in professional environments such as professional sport environments or physical therapy, conventional dynamic body state devices may be unsafe and/or a hassle because the observers don't have an overview of the body state of each individual exercising person.

Other dynamic body state devices, such as running mats, cycling-trainers and cross-trainers have electrodes in handles. To measure the heart rate, these handles have to be firmly held while exercising, oftentimes leading to less comfort and/or strain in the back. For certain higher intensity exercises like running, the arms have to move freely on both sides of the body and holding on handles is just impractical. Furthermore, training coaches for these types of exercises have to pass by the device to see the current state of the sporting person and has to keep in mind the ranges that correspond to the individual needs of each person, for example of the heart rate. This is particularly applicable for persons who need to be monitored because they have a medical condition or are recovering from illness or surgery.

A goal of the invention is to provide a safe and effective dynamic body state device.

This goal and other goals of the invention can be achieved individually or in combination and are not set out in any significant or preferred order.

### SUMMARY OF THE INVENTION

A dynamic body state display device according to the invention is provided, which is wearable by a person on the body and which is at least partly flexible, wherein the device comprises a dynamic body state sensor for measuring the dynamic body state, a storage arrangement for storing information specifying dynamic body state ranges, a processing circuit configured to process the measured dynamic body state and determine the corresponding dynamic body state range, and an at least partly flexible structure, that can be worn on the body, wherein the at least partly flexible structure comprises a display for visually indicating said corresponding dynamic body state range.

### BRIEF DESCRIPTION OF THE DRAWINGS

In clarification of the invention, embodiments thereof will be further elucidated with reference to the drawing. In the drawing:

FIG. 1 shows a schematic drawing of an embodiment of a dynamic body state device;

FIG. 2 shows a general outline of steps in a flow chart of using a dynamic body state device;

FIG. 3 shows an embodiment of a dynamic body state device in use;

FIG. 4 shows an embodiment of a dynamic body state device in use;

FIG. 5 shows a top view of a part of a flexible structure;

FIG. 6 shows a cross section of another flexible structure;

FIG. 7 shows an embodiment of a dynamic body state device;

FIG. 8 shows another embodiment of a dynamic body state device.

### DESCRIPTION OF THE EMBODIMENTS

In this description, identical or corresponding parts have identical or corresponding reference numerals. The exemplary embodiments shown should not be construed to be limitative in any manner and serve merely as illustration.

In this description, dynamic body state refers to a particular body state of a person or animal that changes when exercising, at least when a certain level of effort is made. Dynamic body states for example include but are not limited to heart rate, body temperature, blood oxygenation, amount of body fluids, redness of the skin, but also others among which several are mentioned in this description. Dynamic body state may also include but is not limited to the body state relative to the environment such as acceleration of (parts of) the body, body speed, number of steps, etc. In general it comprises states that can be measured from the outside of the body and which are influenced by exercising. Displaying said states can be used for rehabilitation, safety, sport, amusement and/or fashion purposes and/or other purposes. In preferred embodiments, the dynamic body state is measured using non-invasive methods.

Furthermore, the word ‘range’ should not be considered as limitative to the invention. For example, a range can be an amount or extent of dynamic body states, for example a range between for example 120 and 140 beats/minute. This range is used as a reference value for a dynamic body state device when measuring the dynamic body state.

Furthermore, within the scope of the invention also one dynamic body state number or the like can be applied. For example a heart rate of 140 beats/minute. Hence, when a heart rate below 140 is measured, this can be considered as corresponding to a range between 0 and 140. Any measured heart rate above said 140 could be considered as corresponding to a range that expands from 140 to infinity. In such cases one number may represent two ranges.

In another exemplary embodiment a heart rate sensor measures a heart rate. This heart rate is rounded to a certain number of beats/minute, for example 100 beats/minute, which corresponds to a certain color that is displayed, for example a color having a wavelength of 600 nm. In the same embodiment a heart rate of 101 beats/minute would correspond to another color having a wavelength of 605 nm, for example. Here, one number, for example 101, can be interpreted as corresponding to a certain range, for example from 100.5 to 101.4 beats/minute.

A specific, but non-limiting example of a dynamic body state range is a heart rate range between for example 70 and 90 beats/minute. This illustrative range may for example correspond to a more or less ‘relaxed’ dynamic state of the body. Another illustrative example of dynamic body state range may be a “fat burning” heart rate range between for example 120 and 140 beats/minutes for an overweight person. Yet another illustrative example is a skin temperature range between 39.5° and 40.5° C. which may correspond to a more or less ‘overheated’ physical state, for example. It is mentioned that these and other examples of ranges in this description serve merely as illustrative examples and may depend on circumstances such as the state of the user, the climate, the activity, set of demands, clothing, the embodiment of the dynamic body state device. Any range or combination of ranges may be input in the device, more particularly a storage arrangement 5.

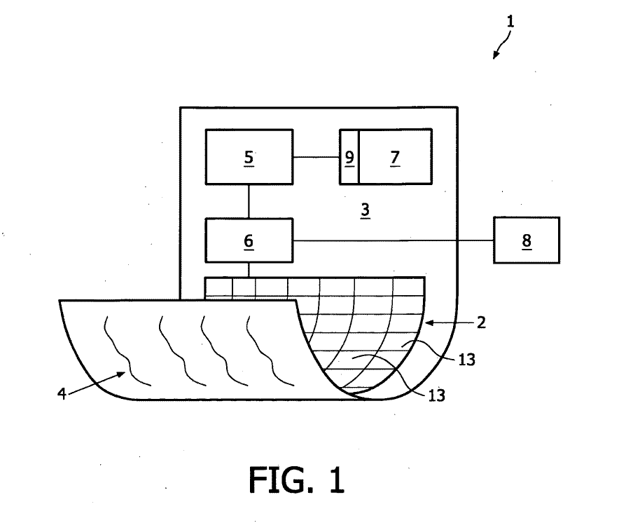

FIG. 1 shows a schematic drawing of an embodiment of a dynamic body state device 1. The device 1 comprises an at least partly flexible, preferably comfortable structure 3 to be worn by a person, for example while this person is exercising. The structure 3 may for example be shaped as a garment like a glove, sleeve, a band/ribbon, or an arm or finger garment. The flexible structure 3 comprises a display 2 comprising LED's 13 (light emitting diodes). When a person exercising wears the device 1 while it is functioning, the display 2 will visually indicate dynamic body state information of this person, based upon input, received from a dynamic body state sensor 4. The display 2 may for example emit a specific color and/or pattern that corresponds to a specific dynamic body state range.

In the embodiment shown in FIG. 1, a dynamic body state sensor 4 is provided at the backside of the device 1 that converts the measured dynamic body state into signals. A storage arrangement 5 is provided wherein information is stored. The information stored in the storage arrangement 5 specifies reference dynamic body state ranges, which ranges are preset in the device 1. Preferably, these ranges are specifically adapted to the user of the dynamic body state device 1. A processing circuit 6 is provided that processes signals that are received from the body state sensor 4, applies certain algorithms, and verifies which of the stored reference dynamic body state ranges corresponds to the dynamic body state that is sensed by the body state sensor 4. The display 2 will indicate the body state by displaying a color and/or a shape that corresponds to the overlapping body state range, preferably in a manner that is clear and easily understandable from a distance, to the outside, i.e. in a direction away from the body.

Furthermore a user interface 8 is provided to be able to set the dynamic body state ranges and at least temporarily store those ranges in the storage arrangement 5. Preferably said ranges are configured for a specific user. For example, for an elderly person in rehabilitation different dynamic body state ranges may be applied than for a professional athlete. The user interface 8 can be attached to and/or integrated with the flexible structure 3 or be provided separately from the flexible structure 3. Also a preferably flexible power supply 7 of any suitable type, such as for example a lithylene battery and/or solar cells, and/or connecting means 9 for the power supply 8 are provided to supply power to the elements of the dynamic body state device 1, such as for example the body state sensor 4, the storage arrangement 5, the display 2 and the user interface 8.

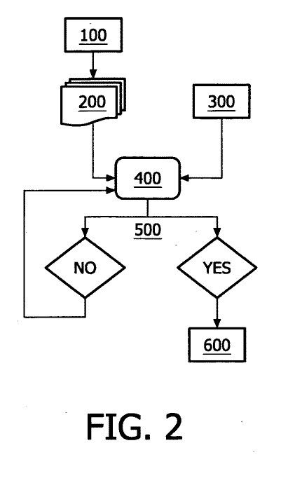

FIG. 2 illustrates a general outline of steps of using an embodiment of a dynamic body state device 1. In step 100, dynamic body state ranges are set, for example through the user interface 8, or during manufacturing. In specific embodiments, different dynamic body state ranges can be set corresponding to different individual needs and/or body conditions. For example, medical and/or cardiovascular tables may be applied to set specific ranges. These ranges can be set by a user, training manager, therapist, specialist, e.g. heart specialist, and/or generally any person. The ranges are brought into correspondence with specific colors and/or patterns to be displayed on display 2. In step 200, those ranges are stored in the storage arrangement 5. In step 300, the dynamic body state device 1 is worn on the body of the user and the dynamic body state sensor 4 measures the dynamic body state, for example while the user is exercising. In step 400, the measured dynamic body state is processed and compared to a first dynamic body state range that corresponds with a specific color and/or pattern of one of the ranges that were set in step 100. Then, at junction 500, if the measured state doesn't fall with the first range, step 400 is repeated for a next range corresponding to a specific color and/or pattern. At junction 500, when the measured dynamic body state corresponds to a specific dynamic body state range, the specific color and/or pattern corresponding to that range is emitted by the display 2, at step 600. The first range that is compared may for example be the range corresponding to the color and/or pattern that is already being emitted by the display 2.

In an embodiment, the LED's 13 in the display 2 are individually addressable to express color codes wherein each color corresponds to a certain dynamic body state range. In principle the LED's 13 or combination of LED's 13 may emit any color. Preferably the LED's 13 at least emit in a range from green to red, e.g. green, amber, red, as these colors correspond to worldly standards, wherein red could indicate a critical body state, yellow, orange or amber could indicate that the critical state is approached, and green could indicate a preferred state, for example, a state that is adapted to that person's condition. For example, green light could be emitted to indicate a heart rate in a range between 70 and 100 beats/minute and red light could be emitted to indicate a heart rate in a range above 150 or below 70 beats/minute.

The LED's 13 could emit in a wavelength range between, but not limited to, 490 to 800 nm, e.g. green, yellow, orange or red. Furthermore, other colors can be used, such as for example blue LED's 13, for example for additional functions or marketing/ornamental purposes. Also other types of LED's 13, such as for example UV (ultraviolet) or blue LED's 13 may be used wherein phosphors, filters and/or other means convert the UV or blue light emitted by the LED's 13 in any desired colored light, which may for example be green, amber and/or red. Preferably, low intensity LED's 13 are used.

In a particular embodiment, and since the LED's 13 are individually addressable, colors are emitted in a particular shape, for example corresponding to a particular dynamic body state, user and/or product brand. Thus, next to information about the dynamic body state of the user, also other information can be communicated, for example advertising. This advertising could also react to the state of the user and/or the environment, wherein different technologies can be used. In another embodiment, RGB LED 13 packages are used, which already contain three color LEDs 13 in a circuit. With appropriate drivers, color mixing of blue, red and green light may be obtained, such that multiple colors may be emitted.

The dynamic body state sensor 4 measures one, multiple or combination of specific dynamic body states such as e.g. heart rate (which may for example also be embodied by pulse rate, ECG (electrocardiogram) and/or EMG (electromiogram)), respiration rate, body temperature (skin or core), heat flow off the body, body fat, hydration level, amount of body fluids, oxygen consumption, energy consumption, redness of the skin, acceleration of the body, body speed, amount of steps, pressure on muscle and/or bone, etc. For example, the sensor 4 can be any type of heart rate sensor, pedometer, accelerometer and/or body temperature sensor. The sensor 4 can for example be separately arranged from the display 2, e.g. the sensor 4 may for example be situated in a shoe or near the pulse, while the display 2 is situated near the back of the user. In those embodiments, input from the sensor 4 to the processing circuit 6 and/or display 2 can be delivered with wireless or physically connected means. In other embodiments, the dynamic body state device 1 is configured, such that sensors 4 are exchangeable.

Different dynamic body states and sensors 4 are mentioned in United States patent application publication US 2006/0122474, which is incorporated herein by reference. In US 2006/0122474, the dynamic body states are referred to as “parameters”. Any of the dynamic body states (i.e. parameters) and/or sensors 4, and/or a combination of states and/or sensors 4, mentioned in the Figs. and description of US 2006/0122474 can in principal be employed in different embodiments of the dynamic body state device 1. For example, it may also be the case that one sensor is configured to measure different dynamic body states. In general, it can be said that the dynamic body state sensor 4 is able to transform a body state measurement into an electronic signal. Other documents that describes sensors 4 for different embodiments of the dynamic body state device 1 include publication numbers US 2004/0100376, US 2006/0100530, US2006/0142658 and WO2005/053532, also herein incorporated by reference.

In an embodiment, heart rate sensors 4 are used. Conventional heart rate sensors 4 may consists of one or more metallic electrodes (stainless steel, copper, gold) or conductive plastic or rubber (e.g. carbon loaded rubber) in contact with the skin on the chest or the wrist. The electrodes measure the “galvanic skin response”. The metallic electrode can for example consist of bulk metal plates or other metallic textile or pad made out of conductive yarns. For example, knitted stainless steel textile electrodes are “dry” and can be an interesting alternative to “gel electrodes”. Different heart rate measurement methods and sensors 4 that can be employed in the dynamic body state device 1 are described in the publications WO2006064447, WO2006067690, EP1494580, EP1578267, WO2004/056268 and US2006/0142658, which are incorporated herein by reference.

In other embodiments, sensors 4 measure blood flow by piezoelectric sensors 4 or Doppler ultrasound, see for example United States patent application publication number US 2006/0135881, which is incorporated herein by reference. Doppler ultrasound techniques measure the frequency shift of the reflected sound, which indicates the velocity of the reflecting material. Other acoustic sensors 4 can also be used, which measure surface acoustic or bulk acoustic waves. These sensors 4 may comprise a coating of thin polymeric or metallic film on top of the sensing surface of a piezoelectric crystal.

Piezoresistive sensors can also be employed in different embodiments of the dynamic body state device 1, for example for measuring movement when placed for example on the shoulder or legs, and for example for measuring respiration rhythm when placed for example on the abdomen or thorax. For example, a known method of the company Milior-Smartex in Italy (www.smartex.it) uses Lycra® coated with carbon loaded rubber and commercial electroconductive yarn as piezoresistive sensors 4. For respiration measurements for example magnetometers, strain or impedance variation can be used. On this subject Dr. R. Paradiso wrote “Wealthy: wearable health care system”, Techtextile symposium North America 2004, herein incorporated by reference.

Other known non-invasive pressure sensors 4 that may be employed in a dynamic body state device 1 are electro-optical sensors, strain gauges and pressure transducers. For example, monitoring a blood pressure signal can be done with strain gauges.

In other embodiments, sensors 4 can be applied that apply optical measurement of the blood through the skin, using appropriate detection methods. In an embodiment of a sensor 4, the blood is illuminated by light of a certain wavelength to detect the presence of certain analytes that are sensitive to that wavelength. This method can for example also be used to measure the oxygenation of the blood, also called pulse oximetry, see also international patent application publication WO 2006/064399, herein incorporated by reference.

In again other embodiments, micro-array sensors 4 are in contact with bodily fluid or bodily vapor (“electronic nose” type) for measuring and are configured to evaluate the presence and/or the quantity of certain analytes. Such sensors 4 can be composed of a layer including a material that can bind to analytes, vapors or markers, whose production or presence may be representative of a certain dynamic body state of the person. Said layer can be coupled to a transducer that transforms the sensor reading into an electronic signal.

In specific embodiments, motion detection sensors 4 detect the appearance of motor seizures (e.g. wherein there may be a partial seizure with localized motor activity, e.g. there may be a spasm or clonus of one muscle or a muscle group and this may remain localized or it may spread to adjacent muscles). An example of such a sensor 4 is an accelerometer.

Sensors can be alone or arranged in arrays. Sensor data processing might involve the use of signal amplification, such as described in international patent application publication WO 2004/056268, herein incorporated by reference.

In general, typical sensors 4 include, but are not limited to, electrodes, piezo elements, temperature sensors, pressure sensors, chemical sensors, and biological sensors. Particular embodiments of the dynamic body state device 1 comprise one main sensor 4, which may be of any type, and have at least one additional sensor 4. For example a heart rate sensor 4 may be applied next a temperature sensor 4, such that next to the heart rate the dynamic body state device 1 also measures the temperature. Obviously, any combination of dynamic body state sensors 4 can be applied. Furthermore the sensors 4 can be applied separately and/or in arrays and/or combination.

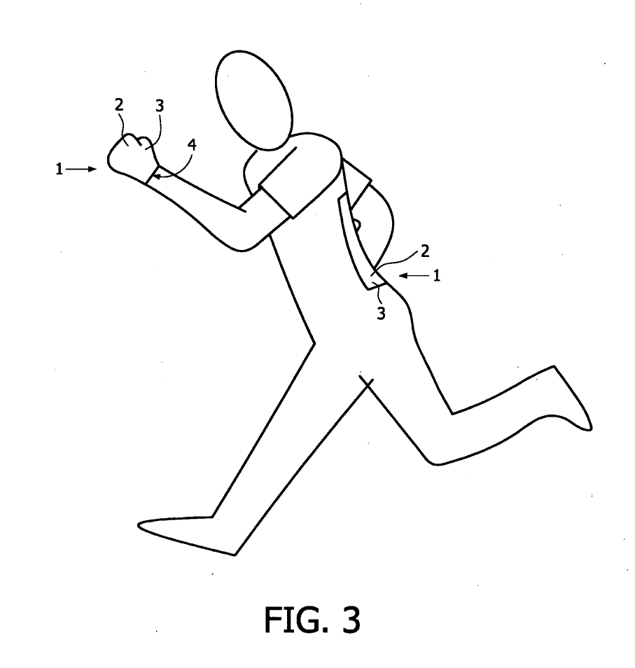

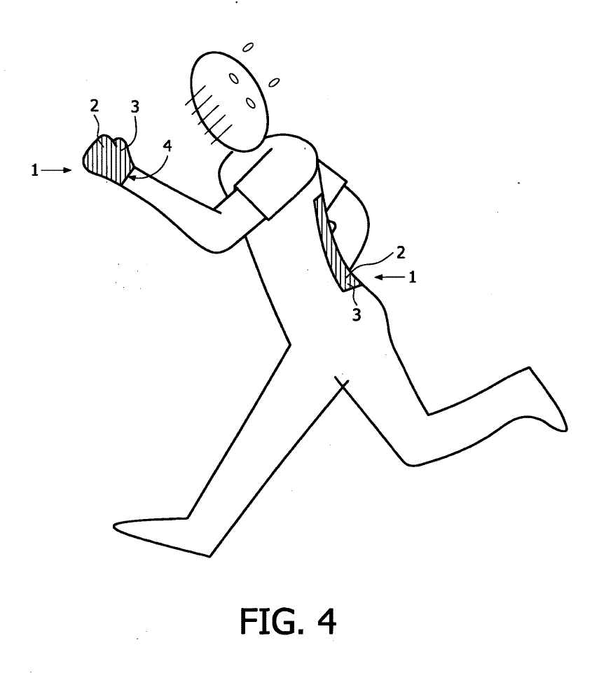

A particular embodiment of the dynamic body state device 1 comprises a heart rate sensor 4 as a main sensor 4 and will therefore be referred to as heart rate device 1. The heart rate device 1 is worn as a garment, as can for example be seen from FIGS. 3 and 4, wherein the garments comprise a glove or a T-shirt or both, depending on the wish of the user. For example, if exercising indoors, the sporter might use only the glove or both glove and T-shirt. In that case the color displayed by glove and T-shirt may be the same. If exercising outdoor, it is recommended to use the T-shirt in any cases, as the visibility of the sporter may be increased in this way. The substantially flexible heart rate device 1 doesn't limit the freedom of usage of the user. In principle, the heart rate device 1 can be worn as any type of garment, or a piece thereof, which may be pulled loose. The display 2 may follow the contour of the body of the user and may be integrated into clothing-like structures 3. For better contact of the sensor 4 to the body, it may be advantageous when the garment is tightly adjusted to the body. Therefore, in particular embodiments, the dynamic body state sensors 4 are integrated with stretch-like textiles 3. The sensors 4 are then pressed again the skin by the elasticity of the sensor 4 as well as the fabric 3. For example, for the comfort of the user, softer fabric pads can be placed between skin and part of the display 2 which could for example be less flexible. In order to further improve the contact between the sensor 4 and the skin, for example hydrogel and/or bodily fluids can be used to make contact.

As already explained, while a person wearing the heart rate device 1 is exercising, for example running, the heart rate sensors 4 will measure the heart rate of that person, whereas the display 2 emits a color, for example green, amber or red, that corresponds to the approximate heart rate. In FIG. 3 the color of the display 2 is green, which may indicate a more or less healthy heart rate. In FIG. 4 the color of the display 2 is red, which may indicate a more or less unhealthy or unadvisable heart rate. The glove, i.e. the heart rate device 1, indicates clearly if it's safe or unsafe to continue the specific exercise with about the same effort. Therefore the person is aware of his or her approximate state of the body, since the glove is clearly in sight and a substantial part is emitting a color. Also observers from a distance, such as for example a training manager and/or a therapist will be aware of the approximate heart rate and will notice a difference. For example, the heart rate device 1 can be advantageous in the field of rehabilitation of patients or for seniors or overweight persons who do not need to exercise at maximum intensity, wherein health care specialist are able to watch over their patients with the aid of the dynamic body state device 1.

Given the size, location of wearing the device 1 on the body, and/or conspicuous colors of the heart rate device 1, the visual signal that is indicated through the display 2 can be seen and interpreted in a glimpse. Even for the exercising user, it may be more effective than a sound signal or a small numerical value on an LCD screen that would require the sporter to bring his wrist closer to his face, breaking the fluidity of his/her sporting movement or rhythm.

To be able to wear the device 1 comfortably and safely, the display 2 comprises a flexible display 2 wherein for example LED's 13 can be integrated in and/or attached to the flexible structure 3, preferably such that the heart rate device 1 may be worn like a garment, as can be seen from FIGS. 3 and 4. Preferably the heart rate device 1 is configured to be of light weight and flat. In different embodiments, the display 2 may be of different shapes and sizes. As a non-limiting example, to be visible from a distance of about 50 meters, a the size of the display may be at least 3×3 cm², approximately. Also smaller or largest sizes, for example of approximately 15×20 cm² can be conceivable. Also the display 2 may for example spread over substantially the whole of the structure 3 and/or dynamic body state device 1, as can be seen from the glove shaped devices 1 in FIGS. 3 and 4.

Embodiments of a dynamic body state device 1, such as for example a heart rate device 1, may be worn as conveniently as any type of garment, wherein the device follows the contours of the body of the user when it is in use, and in use the heart rate is communicated in a way that the rate can be rapidly read and/or read from a relatively large distance. In other words, on the one hand, the heart rate device 1 may function as any type of garment, and on the other hand the heart rate device 1 functions as a conspicuous communicating device wherein in a glimpse the status of the heart rate can be achieved for the observer who is observing multiple users, such as for example a team sports' trainer or coach, or a therapist. With a heart rate device 1 it is possible to provide a global insight of the state of the person wearing the device.

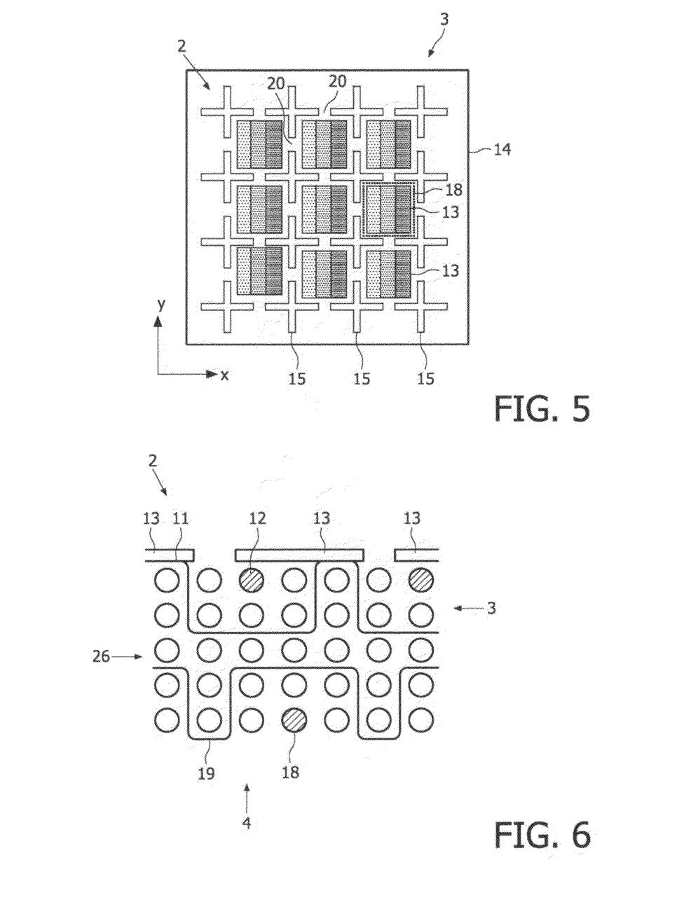

In an embodiment, the structure 3 comprises a flexible foil 14 with LED's 13 attached to it, an example of which is illustrated in FIG. 5. The LED's 13 form a display 2. As can be seen from FIG. 2 the foil 14 is provided with rows and columns of openings 15, between the LEDs 3. Obviously, the foil 14 can be constructed in different shapes and sizes. The openings 15 of the foil 14 increase flexibility and the breathing properties of the dynamic body state device 1, which may be especially useful during exercising and/or for wearing the device 1 as a garment. The sizes and shapes of the openings 15 may also vary, for example depending on the level of flexibility of the foil 14. Different embodiments of the foil 14 for example comprise woven or knitted textiles or polymers, non-woven textiles or polymers, rubbery materials and/or other materials. The foil 14 is provided with not shown conducting lines of an electrical circuit for supplying power to and/or controlling the LEDs 13. In an embodiment, a different set of conducting lines is provided to function as electrodes for the heart rate sensor 4 or any other sensor 4 added to the device 1. The foil 14 with LED's 13, such as for example illustrated in FIG. 5, may form an integral part of the flexible structure 3 or the foil 14 may be attached to a part of the structure 3 as a separate element. In another embodiment, the foil 14 is a separate layer within the structure 3. In some embodiments, the foil display 2 may be detachable from the heart rate device 1. LED's may refer to inorganic LEDs. They may be SMD and low power. They can also be LEDs packages containing three colors: Red, Blue and Green that can be addressed separately, so that color mixing is possible.

In another embodiment the display 2 comprises OLED's (organic LEDs) 13. OLED's 13 are electroluminescent sources that can be configured to be flexible and/or transparent. Also, one OLED 13 or a stacked layer of OLED's 13 can cover a large area of the body without being interrupted, while maintaining flexibility. The device 1 can be provided with one or multiple layers OLED's 13 that may be attached to the flexible structure 3.

In specific embodiments, other display techniques than LED's 13 may be employed in the dynamic body state device 1 instead of or next to LED's 13. Some non-limiting examples are chromatic foils, other thin film electroluminescent devices, phosphor chemicals, flexible neon lights, glass fiber material, liquid crystal display elements (e.g. in cooperation with a suitable light source), plasma, PLED and others. These and other techniques may be specially configured for usage as a display element 2 in a dynamic body state device 1.

An embodiment of the heart rate device 1 is provided with LED's 13 that are attached to yarns 11, 12 in a woven or knitted structure 3 of conducting and non-conducting yarns 11, 12, as can be seen from FIG. 6. These yarns 11, 12 may be at least partly flexible and/or shapeable. The LED's 13 are indicated by dotted lines. These conducting yarns 11, 12 may function as electrodes for said LED's 13, comprising anodes 11 and cathodes 12, wherein the yarns 11, 12 may not be in contact with the skin. In another embodiment, the conducting yarns 11, 12 are electrodes that can function as electrodes for connection of the heart rate sensor 4. In that case, the yarns are also not in contact with the skin, or, for example in contact with the skin via another conductor, such as body fluids and/or hydrogels. As seen previously conductive yarns 18, 19 can also be a part of the heart rate sensor 4 and in that case the yarns 18, 19 are in contact with the body, to measure the heart rate. Preferably, the configuration of the structure 3, more particularly, the yarns 11, 12, is such that the LED's can be individually addressed. The structure 3 can for example comprise textile and/or be combined with other (transparent) layers of textile, structures 3, foil, polymers. for example for extra protection of the body and/or for separate interwoven electrodes for the heart rate sensor 4. Of course, the woven structure 3 can comprise various polymeric textile materials such from natural (cotton, wool), regenerated (viscose) and synthetic fibrous materials such as polyamide (Nylon), polyester, and more, as well as leather or other animal skin, among others. In principle, the structure 3 may be flexible and can have the properties of garments.

Of course, the conducting yarns 11, 12 may be interwoven or knitted in any suitable manner. They can for example also be attached to a textile base by embroidery or printed on the textile material, e.g. conductive inks, dyes or pigments, for example in another manner than is shown in FIG. 6. Conducting yarns 11, 12 may for example be attached to a woven and/or non-woven structure 3 in a non-woven manner. The structure 3 can also contain other layers such as foam or non-woven material, as well as for example spacer fabrics such as diffusive layers in order to produce homogenous light emitting areas out of point sources of light such as LEDs 13. The structure 3 can also have an elastomer transparent cover layer for robustness in its entirety or for each individual LED 13. In different embodiments, the structure 17 comprises woven or non-woven fabric or textile, a polymer or rubbery substance. Embodiments of ‘non-woven’ structures 3 comprise fibers that are bonded together, for example by use of resins or mechanical entanglement, as compared to woven fabric, where the fibers may be held together by weaving of the yarn and/or twisting of the fibers in the yarn. Suitable fibers include natural textile fibers, such as cotton or wool fibers, regenerated fibers, such as viscose, and synthetic fibers such as polyester, polyamide (nylon) or polyacrylic fibers. The structures 3 may also comprise at least partly transparent protection layers for on top of the display 2, for example for protection from bumping and/or wet surroundings.

Certain embodiments of the heart rate device 1 are configured to indicate the presence of the wearer of the device 1 in the dark, for example, because many times outdoor sport's persons aren't noticed by automobile drivers when it's dark outside. For example, an additional flashing signal can be produced. This flashing signal can help in indicating the presence of the user, but may also be of use to indicate a warning signal via the display 2 when a specific heart rate range is reached. For example, in indicating the presence of the user, it is preferable that the back of the T-shirt does not display a green signal but another signal, such as red, so that there is no confusion in the mind of the automobile driver that he has to stop preventively.

In an embodiment, such as the exemplary embodiment shown in FIG. 6, the heart rate sensor 4 comprises conducting yarns 18, 19. The conducting yarns 18, 19 can be integrated with any structure 3, e.g. woven and non-woven structures 3, in more or less the same way as conducting yarns 11, 12 that function as electrodes for the LED's 13 that have already been described. The conductive yarns 11, 12 of the LED display 2 and the conductive yarns 18, 19 of the sensor 4 can be separated, for example on two different textile layers separated by an insulating textile layer 26, or could be arranged on different sides of the dynamic body state device 1.

In specific embodiments the heart rate sensor 4 comprises a circuit that emits an oscillating magnetic field for measuring the conductivity in a volume (such as the body), wherein the magnetic field is created from a conductive coil integrated in an insulating fabric, such as is the case with the earlier mentioned patent application publication US 2006/0142658. With such a sensor 4, a sensitive and mechanically flexible sensor 4 is obtained. The conductive coil in interwoven in with threads of fabric.

An embodiment of a heart rate device 1 comprises a user interface 8 through which individual preferences for the heart rate device 1 may be set. These settings are stored in the storage arrangement 5. Any user interface 8 can be suitable. For example, the user interface 8 may comprise a mechanical, electrical and/or digital set-up. In some embodiments, the user interface is also made flexible.

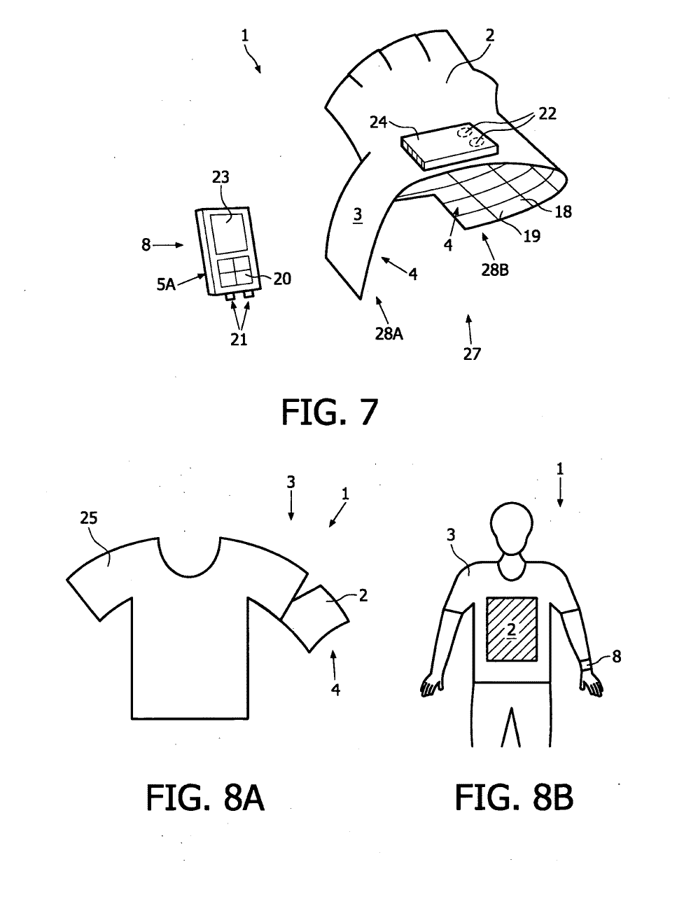

In FIG. 7, an embodiment is illustrated, wherein a user interface panel 8 is provided. The user interface panel 8 comprises input elements 20, connector elements 21, an LCD screen 23 and a panel storage arrangement 5A (comprising a circuit that may be inside the housing of panel 8) for storing information that has been set through the user interface panel 8. The panel storage arrangement 5A is configured to store information specifying body state ranges, like for example heart rate ranges. The separate user interface panel 8 can be connected or make connection to the rest of the heart rate device 1. In an exemplary embodiment, as can be seen from in FIG. 7, the heart rate device 1 is configured to be worn like a glove 27, wherein in the shown embodiment the fingers of the glove 27 are cut off When a user wants to use the heart rate device 1 he makes a connection between the glove part 27 of the heart rate device 1 and the user interface element 20, for example by connecting the connectors 21 and 22, so that the individual settings from the storage arrangement 5A of the user interface element 8 are uploaded to the storage arrangement 5 of the glove part 27 of the heart rate device 1. In use, the dynamic body state device 1 may refer to the storage arrangement 5. Obviously, different configurations of one and/or multiple storage arrangements 5 and/or 5A are possible within the scope of the invention.

Said glove part 27 and user interface panel 8 may for example be connected through wireless information sharing. As can be seen from FIG. 7 the glove part 27 of the device 1 comprises a flexible structure 3, a display 2 and a heart rate sensor 4 comprising conducting yarns 18, 19. These conducting yarns 18, 19 may in an embodiment be configured to contact the skin near the pulse of the user, such that for example pulse rate can be measured in an advantageous way. For example, near the pulse, the glove part 27 may be tightened in an advantageous way by attaching a first tightening element 28A of the glove 27 to a second tightening element 28B, such that the yarns 18, 19 may contact the skin tightly. Said tightening elements 28A, 28B may for example comprise Velcro®.

In a particular embodiment, the user interface panel 8 is in some way attachable to the glove part 27 of the device 1. As can be seen from FIG. 7, an arrangement 24 is provided to carry the user interface panel 8 and/or make a connection through connectors 21 and 22, so that individual settings can be read from the user interface panel 8 wherein a separate storage is provided.

The user interface panel 8 may for example also be a memory stick and/or memory card that can be put in the garment. With this embodiment, specific settings may for example be applied and stored before usage. These settings can be uploaded by a personal computer at home, or some dedicated device, during manufacturing/programming.

In FIG. 8A, an embodiment of a heart rate device 1 is shown that is modular. The flexible structure 3 of the heart rate device 1 is shaped as a garment, in particular a T-shirt. A piece of the structure 3 can be separated from the device 1, for example a part that has circuitry, so that the other part of the device 1, in FIG. 8A indicated with reference number 25, can be washed separate from the circuitry. In other embodiments, the whole of the heart rate device 1 including the circuits is of modular shape, i.e. separate parts of for example the display 2, can be separated from each other and connected again, for example to be able to measure different body parts.

In another embodiment a T-shirt shaped heart rate device 1, such as can be seen from FIG. 8A, can be connected to a glove shaped device 1, such as can be seen from FIG. 7. This is shown in FIG. 8B. For example, the T-shirt may at least comprise a display 2, whereas the glove at least comprises the user interface 8 and the sensor 4. In an embodiment, the T-shirt and glove parts communicate through wireless communication.

Although the above description will mainly describe embodiments wherein the dynamic body state sensor 4 comprises a heart rate sensor 4, the invention should not be limited to this. Although certain embodiments have been discussing a heart rate device 1, the same principles could be applied to other embodiments of dynamic body state devices 1. A person skilled in the art will be able to translate the relevant functions of the heart rate device 1 to dynamic body state devices 1 with one or a combination of said other sensors 4.

Furthermore, it should be considered that the invention is not limited to the field of health, sports and/or rehabilitation. but could also be applied in other fields. The product may for example also be applied as a specific type of life style and/or advertising element and/or be incorporated into shoes, caps, etc.

Applications and/or conditions for use of the invention may involve, but are not limited to indoor or outdoor exercising, rehabilitation after surgical operation (also including sportsmen in recovery, for example coupled with muscle strain sensors), stroke recovery, cardio-vascular accident recovery, people that would need to be monitored for a certain period of time like babies or sick infants, or older people in retirement homes if they present a particular risk, for example with respect to cardiac events. Overweight people who need to exercise between 120 and 140 beats/minute (low-intensity fat burning) may also profit from the invention. Another application for the invention may involve asthma (for example, using a respiration sensor).

It shall be obvious that the invention is not limited in any way to the embodiments that are represented in the description and the drawings. For example, the invention should not be limited to the sensors 4 that are mentioned in the description, since other sensors 4 may also be applied within the scope of the invention. Many variations and combinations are possible within the framework of the invention as outlined by the claims. Combinations of one or more aspects of the embodiments or combinations of different embodiments are possible within the framework of the invention. All comparable variations are understood to fall within the framework of the invention as outlined by the claims.

1. Dynamic body state display device (1), which is wearable by a person on the body and which is at least partly flexible, wherein the device comprises a dynamic body state sensor (4) for measuring the dynamic body state, a storage arrangement (5) for storing information specifying dynamic body state ranges, a processing circuit (6) configured to process the measured dynamic body state and determine the corresponding dynamic body state range, and an at least partly flexible structure (3), that can be worn on the body, wherein the at least partly flexible structure comprises a display (2) for visually indicating said corresponding dynamic body state range.

2. Device according to claim 1, wherein the display (2) comprises LED's (light emitting diodes).

3. Device according to claim 1, wherein the display (2) is at least partly flexible.

4. Device according to claim 1, wherein the dynamic body state comprises a heart rate sensor.

5. Device according to claim 1, wherein the dynamic body state sensor comprises electrodes.

6. Device according to claim 1, wherein at least one sensor is configured to measure at least two different dynamic body states.

7. Device according to claim 1, wherein the structure comprises a woven and/or knitted and/or otherwise bonded structure of textile and/or synthetic textile material and/or leather or animal skin.

8. Device according to claim 1, wherein at least part of said structure is a separate module that can be attached to and separated from the device.

9. Device according to claim 1, wherein the device comprises electrodes that are conductive yarns, preferably interwoven with, knitted and/or bonded to the device, preferably for connecting the heart rate sensor and/or for driving the LED's.

10. Device according to claim 1, wherein the display comprises a foil with LED's of at least two different colors.

11. Device according to claim 1, wherein the display comprises organic LED's of at least two different colors.

12. Device according to claim 1, wherein the display comprises blue and/or ultraviolet LEDs and phosphors, wherein the phosphors are configured to transform the color of the LEDs into at least one color that is different from the original color of said LEDs.

13. Device according to claim 1, wherein the display comprises LED packages that have red, green and blue LEDs, which are configured to emit multiple colors.

14. Device according to claim 1, wherein the display is configured to emit light approximately in the green range, approximately in the yellow or orange range and approximately in the red range.

15. Device according to claim 1, wherein the device comprises a user interface for setting dynamic body state ranges.

16. Device according to claim 15, wherein the user interface is separately provided together with a second storage arrangement for storing information specifying dynamic body state ranges.

17. Garment, comprising a device according to claim 1.

18. Garment, comprising a combination of garments according to claim 17 and/or parts of a garment according to claim 17, wherein the garments or parts of a garment can be physically connected and disconnected.

19. Garment according to claim 18, wherein elements of the dynamic body state device are divided over said garments or parts of a garment.

20. Method of showing a dynamic body state, wherein at least one reference range that corresponds to dynamic body states of a specific user is stored, wherein a dynamic body state of that user is measured, wherein the measured dynamic body state is compared to the at least one reference range, wherein the reference range that corresponds with the measured state is displayed over an at least partly flexible surface of a garment.
